(19) United States
(12) Patent Application Publication
(10) Pub. No.: US 2006/0171933 A1
(43) Pub. Date: Aug. 3, 2006

(54) Cell culture

(76) Inventor: Robert Short, Sheffield (GB)

Correspondence Address: KLARQUIST SPARKMAN, LLP, 121 SW SALMON STREET, SUITE 1600, PORTLAND, OR, 97204, US

(21) Appl. No.: 10/548,236

(22) PCT Filed: Mar. 3, 2004
(86) PCT No.: PCT/GB04/00881

§ 371 Date: Sep. 2, 2005

(30) Foreign Application Priority Data
Mar. 5, 2003 (GB) 0304918.6

(51) Int. Cl. A61K 48/00; C12N 5/08

(52) U.S. Cl. 424/93.21; 435/366

(57) ABSTRACT

A method for culturing mammalian cells without the need for the addition of xenobiotic materials which promote mammalian cell culture, for example, serum or a pituitary extract and including methods for the production of cells for use in tissue engineering and the production of recombinant protein.

The invention relates to a method for culturing mammalian cells without the need for the addition of xenobiotic materials which promote mammalian cell culture, for example, serum or a pituitary extract.

The culturing of mammalian cells has become a routine procedure and cell culture conditions which allow cells to proliferate are well defined. Typically, cell culture of mammalian cells requires a sterile vessel, usually manufactured from plastics, defined growth medium and, in some examples, feeder cells and serum, typically calf serum. The feeder cells function to provide mitogenic signals which stimulate cell proliferation and/or maintain cells in an undifferentiated state. The feeder cells are typically fibroblasts which have been treated such that the fibroblasts cannot proliferate (e.g. mitomycin, irradiation treatment, or less typically through the use of a media in which fibroblasts cannot proliferate e.g. a low calcium media). Typically feeder fibroblasts are murine in origin (as in Rheinwald and Green, 1975 Rheinwald J, Green H, Serial cultivation of strains of human epidermal Keratinocytes: the formation of colonies from single cells, Cell, 1975, Vol 6, pp 331-344.).

It would be advantageous if cell culture conditions could be established which did not require the addition of xenobiotic materials such as bovine serum or pituitary extract or murine cells since their use increases the likelihood of infectious agents (e.g. viruses and prions, in particular for bovine products, and murine viruses for mouse feeder cells) infecting mammalian cells grown in culture. With respect to feeder cells it would be advantageous also if autologous fibroblasts could be used as a feeder layer and that these could be growth arrested without the use of mitomycin C or irradiation treatment.

Tissue engineering is an emerging science which has implications with respect to many areas of clinical and cosmetic surgery. More particularly, tissue engineering relates to the replacement and/or restoration and/or repair of damaged and/or diseased tissues to return the tissue and/or organ to a functional state. For example, and not by way of limitation, tissue engineering is useful in the provision of skin grafts to repair wounds occurring as a consequence of: contusions, or burns, or failure of tissue to heal due to venous or diabetic ulcers. Tissue engineering requires in vitro culturing of replacement tissue followed by surgical application of the tissue to a wound to be repaired. To increase the likelihood that the in vitro generated tissue is free from infectious agents (e.g. viruses, mycoplasma, prions) it would be desirable to reduce or avoid exposure of tissue to xenobiotic agents which may be present in serum, pituitary extract or xenobiotic cells.

The cell-types which are typically cultured in vitro for subsequent use in tissue engineering include, by example and not by way of limitation embryonic and adult stem cells (e.g. embryonic and germ cell stem cells derived from human embryos, so called pluripotential stem cells and adult stem cells such as haemopoietic stem cells from which are derived cells which comprise blood, e.g. T-lymphocytes (helper and killer), B-lymphocytes) and adult differentiated cells which can be maintained in culture (e.g. fibroblasts, keratinocytes).

The ability to produce cell cultures in the absence of xenobiotic materials has many other applications. The large scale production of recombinant protein requires a high standard of quality control since many of these proteins are used as pharmaceuticals, for example: growth hormone; leptin; erythropoietin; prolactin; TNF, interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukemia inhibitory factor (LIF); oncostatin M (OSM); interferon, IFNα and IFNγ. Moreover, the development of vaccines, particularly subunit vaccines, (vaccines based on a defined antigen, for example gp120 of HIV), requires the production of large amounts of pure protein free from contaminating antigens which may provoke anaphylaxis.

Seruin free culture of mammalian cells is known in the art. For example, WO98/08934 discloses a cell culture medium that supports in vitro cultivation of mammalian cells such as epithelial cells or fibroblasts. The media comprises a basal medium to which is added a polyanionic compound, for example dextran sulphate. Other attempts have made to avoid the use of animal derived growth factors by using non-animal peptide supplements, for example yeast cells in a basal media. Wheat gluten extracts have also been used to culture mammalian cells (see JP2-49579). However, none of these approaches provide optimal growth conditions for the culture of mammalian cells

The present invention relates to a cell culture system that can maintain cells in culture in the absence of xenobiotic or allogenic agents, for example serum, pituitary extract. We describe a method to culture mammalian cells which involve the simple addition of feeder cells, for example fibroblasts, to a cell culture vessel, in the absence of serum. Surprisingly, we have found that in the absence of serum in the culture media, that a number of different cell types (e.g. fibroblasts, osteoblasts, keratinocytes) will attach and spread on a range of substrata (tissue culture plastics, plasma polymerised films of octadiene monomer) on which they would not normally attach in the presence of serum (Ros Daw, PhD Thesis, University of Sheffield, 1998; John Kelly, PhD Thesis University of Sheffield, 2001; Michael Higham, unpublished data, University of Sheffield). Although, the spreading of these cells is irregular and without serum these cells cannot divide (i.e. mitosis is inhibited), they remain metabolically active and can positively influence both cell attachment and cell proliferation. Typically, cells are seeded onto this feeder layer. The fibroblasts provide both soluble factors (in their conditioned media) and insoluble factors (in the extracellular matrix material they produce) which promote cell attachment and proliferation.

According to an aspect of the invention there is provided a method to culture mammalian cells comprising providing a cell culture vessel which includes feeder cells and growth media, wherein said growth media does not include growth promoting agents which would typically be required for the establishment of a mammalian cell culture; providing conditions sufficient to allow said feeder cells to provide agents which promote mammalian cell culture and providing said mammalian cells in said vessel the culturing of which is desired.

According to an aspect of the invention there is provided a method for the culture of mammalian cells comprising the steps of:

i) providing a cell culture vessel comprising a cell culture support surface comprising feeder cells and cell culture media which does not include agents which promote or enhance the establishment of mammalian cells in culture;

ii) providing culture conditions which promote the production by said feeder cells of agents which promote mammalian cell culture; and

iii) adding to said vessel at least one mammalian cell the culturing of which is desired.

In a preferred method of the invention said agent which promotes mammalian cell culture is derived from serum.

In an alternative method of the invention said agent which promotes mammalian cell culture is derived from a pituitary extract.

In a preferred embodiment of the invention said feeder cells are stromal cells.

Preferably said stromal cells are provided as a cell composition comprising fibroblasts (from any source-eg dermal or oral), dermal papilla cells, chondrocytes, osteoblasts, endothelial cells, astrocytes and keratocytes.

In a further preferred method of the invention said feeder cells are fibroblasts.

In a further preferred method of the invention said feeder cells are epithelial cells. For example human embryonic kidney cells, such as cell line 293, which are particularly useful in the expression of recombinant protein.

The invention includes other combinations of cells which in vivo act as support cells supplying trophic signals to more specialised differentiated cells. A further example of this would be autologous fibroblasts or epithelial cells acting as a feeder layer to support the survival and expansion of cancer cells required for the diagnosis or treatment of patients, e.g. when tumour cells are cultured with cells of the immune system under conditions designed to induce a host immune response when cells (e.g. tumour infiltrating lymphocytes) are reintroduced to the patient. Preferably said feeder cells are human.

The invention also includes genetically engineered feeder cells which are adapted to manufacture agents, typically growth factors, which promote mammalian cell culture.

In a preferred method of the invention said mammalian cells are human.

In a further preferred method of the invention said mammalian cells are selected from the group consisting of: dermal or oral fibroblasts; epidermal or oral keratinocytes adult skin stem cells; embryonic stem cells; melanocytes, corneal fibroblasts (known as keratocytes), corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

In a preferred method of the invention said mammalian cells are keratinocytes, preferably autologous keratinocytes.

It will be apparent to the skilled artsan that it may be necessary to add other supplements to support mammalian cell culture in absence of serum. Cinatl et al (1992) “Protein free culture of Vero cells: A substrate for replication of human pathogenic viruses”, Cell Biol. Int. 17, 885-895 describes a serum free medium with 97 supplements. WO 98/04680 describes serum free medium comprising basal media with 25 or 26 supplements. Supplements are known to those skilled in the art and include growth factors (e.g. fibroblast growth factor), recombinant proteins (insulin, transferrin), salts and vitamins. The use of a feeder layer may replace the need for some (e.g. fibronectin to condition the plastic surface for cell attachment) or all of these supplements. These supplements are commercially available from a number of sources, for example see Sigma Aldrich at http://www.sigmaaldrich.com/

In a further preferred method of the invention said vessel is selected from the group consisting of: a petri-dish; cell culture bottle or flask; multiwell plate. “Vessel” is construed as any means suitable to contain a mammalian cell culture.

In a preferred method of the invention said vessel comprises a non-porous polymer. Preferably a solid-phase substrate, e.g. plastics, glass, contact lenses. Plastics used in the manufacture of cell culture vessel products include polyethylene terephthalate, high density polyethylene, low density polyethylene, polyvinyl chloride, polypropylene. Typically, said vessel are manufactured from polystyrene.

The plastics used in cell culture are typically manufactured from polystyrene and are surface treated to improve cell adhesion/attachment. Suitable surface treatments include alkali/acid rinses, flame or corona treatment and plasma treatment. The latter may involve the use of an inert gas (e.g. argon) or an inert gas/reactive gas mixture (argon/oxygen) or reactive gas (oxygen, air etc.) In very specific cases a nitrogen containing reactive gas (e.g. ammonia) may be used. Biomolecule coating of the plastic (e.g. collagen, or gelatin) are often required for growth of specific cell types (e.g. keratinocytes(collagen) and endothelial cells (gelatin).

In a further preferred method of the invention said feeder cells, for example fibroblasts, are non-proliferative.

In a further preferred method of the invention feeder cells are rendered non-proliferative by a method which avoids the use of mitomycin C or irradiation. Another approach is to provide a media which permits the growth of epithelial cells in co-culture but inhibits or prevents the growth of fibroblast feeder cells. Typically, calcium levels could be reduced to about one-tenth physiological levels to achieve this effect.

In a further preferred method of the invention said feeder cells are human fibroblasts, preferably human dermal fibroblasts. A further source of feeder cells are oral fibroblasts.

According to a further aspect of the invention there is provided a method to culture mammalian cells on a therapeutic vehicle comprising the steps of:
i) providing a preparation comprising a therapeutic vehicle comprising a substrate and attached thereto feeder cells; and cell culture media wherein said media does not include agents which promote or enhance the establishment of mammalian cells in culture;
ii) providing culture conditions which promote the production by said feeder cells of agents which promote mammalian cell culture; and
iii adding to said preparation at least one mammalian cell the culturing of which is desired on said vehicle.

In a preferred method of the invention said mammalian cells are human.

In a further preferred method of the invention said mammalian cells are selected from the group consisting of epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

Preferably said mammalian cells are autologous, preferably autologous keratinocytes.

In a further preferred method of the invention said fibroblast feeder cells are human.

In a further preferred method of the invention said fibroblast feeder cells are human dermal fibroblasts or human oral fibroblasts. Preferably said feeder cells are autologous.

In a further preferred method of the invention said therapeutic vehicle is selected from the group consisting of: prosthesis; implant; matrix; stent; biodegradable matrix; polymeric film or polymeric or natural matrix (e.g. chitin) particles for achieving suspension culture.

Therapeutic vehicles which are manufactured from porous and fibrous materials, woven and non-woven materials, are also within the scope of the invention (e.g. bandages, gauze, plaster casts, tissue engineering scaffolds e.g. PGA/PLA scaffolds).

The initial attachment of cells to polymer therapeutic vehicles, porous and non porous, woven and non woven, biogradable, films and scaffolds, would be enhanced by methods of surface treatment, which would increase the surface hydrophilicity of the material, or introduce new functional groups. These methods are known to those skilled in the art and include plasma treatment (inert gas, air, water, oxygen, nitrogen, ammonia or combinations thereof), corona disharge, flame treatments or simple acid and alkali washes. Numerous publications describe these methods, for example see Biomaterial Science: An Introduction to Materials in Medicine, B D Ratner, A S Hoffman, F J Schoen, J E Lemons, Academic Press, 1996.)

The direct culturing of mammalian cells on a therapeutic vehicle under conditions herein disclosed has obvious benefits in tissue engineering since the fabrication of the surface of said vehicle allows both culturing, implantation and transfer of cells to a wound to be repaired with ease.

According to an aspect of the invention there is provided a method for the production of recombinant protein comprising:

i) providing a cell culture vessel comprising a cell culture support surface comprising feeder cells and cell culture media which does not include agents which promote or enhance the establishment of mammalian cells in culture;

ii) providing culture conditions which promote the production by said feeder cells of agents which promote mammalian cell culture; and

iii) adding to said vessel at least one transfected mammalian cell the culturing of which is produces said recombinant protein.

In a preferred method of the invention said recombinant protein is a therapeutic protein.

In a preferred method of the invention said therapeutic protein is a cytokine Preferably said cytokine is selected from the group consisting of: growth hormone; leptin; erythropoietin; prolactin; TNF, interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukaemia inhibitory factor (LIF); oncostatin M (OSM); interferon, IFNα and IFNγ.

In an alternative preferred embodiment of the invention said therapeutic protein is an antigenic polypeptide.

An embodiment of the invention will know be described by example only and with reference to the following materials and methods.

Materials and Methods

Culture substrates tested include “as received” tissue culture plastic (Iwaki, UK), bacteriological grade plastic, plasma polymerised octadiene surface and a collagen-coated plastic. Collagen coated tissue culture plates were prepared by air-drying a solution of collagen I (32 μg cm⁻²) in 0.1M acetic acid (200 ug ml⁻¹) in a laminar flow cabinet overnight.

X-ray Photoelectron Spectroscopy (XPS) Analysis

XPS was performed using a VG CLAM 2 spectrometer with Mg Kα X-ray source operating at a power of 100W. The spectrometer was calibrated using the Au 4f 7/2 peak position at 84.00 eV and the separation between the C 1s and F 1s peak positions in a sample of PTFE measured at 397.2 eV, which compares well with the value of 397.19 eV reported by Beamson and Briggs [Beamson G and Briggs D, High Resolution XPS of Organic Polymers: The Scienta ESCA300 Handbook, 1992, John Wiley and Sons Chichester]. Spectra were acquired using a fixed take off angle of 30° with respect to the sample surface using Spectra 6.0 software (R.Unwin Software, Cheshire, UK). A wide scan (0-1100 eV) and narrow scans of each sample were acquired. Wide scans were used to obtain the surface oxygen/carbon (O/C) ratio and the narrow scans used to obtain information on the carbon, oxygen and nitrogen binding environments. For the collection of spectra for the wide and narrow scan, the analyser pass energies used were 50 and 20 eV respectively.

ESCA300 (Scienta Software) was used to obtain the peak fits of the C1s core level spectra Gaussian-Lorenzian (G/L) peaks of mix 0.8-0.9 were fitted to the C 1s core level spectrum using well-established chemical shifts [Beamson and Briggs]. In the peak fitting, the full width half maximiums (FWI) of the peaks were kept equal and in the range of 1.38 to 1.67. A hydrocarbon peak was set to 285 eV to correct for any sample charging. Sample charge was in the region of 4-5 eV.

Cell Culture

Human dermal fibroblasts were obtained from the dermal layer of the skin after trypsinisation of a split-thickness skin graft, which was taken from specimens following routine surgery procedures (breast reduction and abdominoplasty), following washing in PBS and then minced finely with a scalpel and placed in 0.5% collagenase. Following centrifugation of the collagenase digest and elimination of the supernatant, the cells were resuspended in 10 mls of fibroblast culture medium (FCM) in a T25 Flask. The flask is maintained at 37° C. in a 5% CO₂ atmosphere.

Human oral fibroblasts were obtained from biopsies of oral mucosa from specimens obtained from patients undergoing routine oral surgery. Material used was that which would otherwise be discarded and was used with the consent of patients. Fibroblasts were obtained and cultures as for dermal fibroblasts as described above.

Every 500 ml of FCM consists of 438.75 mls of Dulbecco's Modified Eagle's medium (DMEM), 50 mls of Foetal Calf Serum (FCS) [optional—see below], 5 mls of 1-Glutamine, 5 mls of Penicillin/Streptomycin (10,000 U/ml and 10,000 ug/ml respectively), 1.25 mls of Fungizone.

FCM without FCS contains an additional 50 mls DMEM to compensate and Insulin (100 ng/ml) and basic fibroblast growth factor (bFGF) (100 ng/ml). (Both Insulin and bFGF are recombinant proteins not sourced from animal tissues).

Fibroblast cells were passaged when 90-100% confluent and used between passage numbers 5 and 9. While comparing the attachment of fibroblasts to culture substrates with and without FCS, the same flask and passage number of cells was employed. Passaging of the fibroblasts was achieved using 1.5 ml of a 1:1 mixture of 0.1% trypsin and 0.02% EDTA per T25 flask.

Human epidermal keratinocytes (obtained from breast reductions and abdominoplasties) were freshly isolated from the dermal/epidermal junction.

Green's media which is routinely used in the culture of keratinocytes includes cholera toxin (0.1 nM), hydrocortisone (0.4 μgm⁻¹), EGF (10 ngm⁻¹), adenine (1.8×10⁻⁴M), tri-iodo-L-thyronine (2×10⁻⁷ M), fungizone (0.625 μg ml⁻¹), penicillin (1000 IU ml⁻¹), streptomycin (1000 μg ml⁻¹) and 10% foetal calf serum (optional). Cells were cultured at 37° C. in a 5% CO₂ atmosphere.

In co-culture experiments, where fibroblasts act as a feeder layer for the keratinocytes, the fibroblasts were seeded at a density of ca. 2×10⁴ cells/ml in DMEM with and without serum for 24 hrs prior to the addition of keratinocytes. On the addition of keratinocytes, the media was removed and keratinocytes were seeded at a density of ca. 2×10⁴ cells/ml, in Green's media with and without serum. In these experiments, collagen I acts as a positive control surface as well as a demonstration that culture can be carried out on a xenobiotic surface without the addition of serum or pituitary extract.

Assessment of Cell Attachment Viability and Proliferation

For investigation of human dermal fibroblast attachment and viability, cells were seeded at a density of ca. 7 10³ cells ml⁻¹ into 24 separate well plates (1.6 cm diameter). Human epidermal keratinocytes were seeded at a density of ca. 4×10⁵ cells/ml. Co-culture experiments used a keratinocyte seeding density of ca. 1.5×10⁵ cells/ml with irradiated dermal fibroblasts at 2×10⁴ cells/ml, irradiated for 4780 seconds using a Caesium 137 sealed source.

The attachment and viability of cells at time points upto seven days were assessed using an MTT-ESTA assay. This assay indicates viable cells and provides an indirect reflection of cell number, in that the cellular de-hydrogenase activity, which converts the MTT substrate to a coloured formazan product, normally relates to cell number.

Cells were washed with 1 ml of PBS solution and then incubated with 0.5 mg ml⁻¹ of MTT in PBS for 40 minutes. 300 μl of acidified isopropanol was then used to elute the stain. 150 μl was then transferred to a 96 well plate. The optical density was read using a plate reader set at a wavelength of 540 nm with a protein reference of 630 nm subtracted. In addition, the appearance of the cells was assessed and recorded at the same time points.

The DNA content of the cells (which reflects cell number but not necessarily viability) was calculated at the same time periods using a Hoechst fluorescent stain (33258 Sigma Chemicals). Cells were incubated in 1 ml of digestion buffer for 1 hour. This buffer consisted of 48 g urea, which breaks up the cells and 0.04 g of Sodium Dodecyl Sulphate (SDS), which protects the cells from DNase, per 100 ml of saline sodium citrate (SSC). Following digestion, cells were stained using the Hoechst fluorescent stain, in an SSC buffer at 1 μg/ml. A fluorimeter was used to measure the fluorescence using excitation and emission wavelengths of 355 and 460 nm respectively. A standard curve of known DNA concentrations was used to calculate the DNA content. For all experimental data presented, cells cultured on their own or in co-culture for upto seven days had a fresh change of media at day three.

Statistics

The significance of an irradiated fibroblast feeder layer in improving keratinocyte proliferation with and without serum was analysed using a statistical two-tailed Student t test where values of p<0.05 were considered as statistically significant.

1. A method for the culture of mammalian cells comprising the steps of:
i) providing a cell culture vessel comprising a cell culture support surface comprising feeder cells and cell culture media which does not include agents which promote or enhance the establishment of mammalian cells in culture;
ii) providing culture conditions which promote the production by said feeder cells of agents which promote mammalian cell culture; and
iii) adding to said vessel at least one mammalian cell the culturing of which is desired.

2. A method according to claim 1 wherein said agent which promotes mammalian cell culture is derived from serum.

3. A method according to claim 1 wherein said agent which promotes mammalian cell culture is derived from a pituitary extract.

4. A method according to claim 1 wherein said feeder cells are stromal cells.

5. A method according to claim 4 wherein said stromal cells are provided as a cell composition comprising: fibroblasts, dermal papilla cells, chondrocytes, osteoblasts, endothelial cells, astrocytes and keratocytes.

6. A method according to claim 1 wherein said feeder cells are fibroblasts.

7. A method according to claim 1 wherein said feeder cells are epithelial cells.

8. A method according to claim 1 wherein said feeder cells are genetically engineered feeder cells.

9. A method according to claim 1 wherein said feeder cells are human.

10. A method according to claim 1 wherein said mammalian cells are human.

11. A method according to claim 1 wherein said mammalian cells are selected from the group consisting of: fibroblasts; epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts,_oral mucosa keratinocytes, urethral fibroblasts and epithelial cells, bladder fibroblasts and epithelial cells, neuronal glial cells and neural cells, hepatocyte stellate cells and epithelial cells.

12. A method according to claim 11 wherein said mammalian cells are keratinocytes.

13. A method according to claim 12 wherein said keratinocytes are autologous.

14. A method according to claim 11 wherein said mammalian cells are fibroblasts.

15. A method according to claim 14 wherein said mammalian cells are autologous.

16. A method according to claim 1 wherein said vessel is selected from the group consisting of: a petri-dish; cell culture bottle or flask; multiwell plate.

17. A method according to claim 16 wherein said vessels are manufactured from plastics selected from the group consisting of: polyethylene terephthalate, high density polyethylene, low density polyethylene, polyvinyl chloride, polypropylene and polystyrene.

18. A method according to claim 1 wherein said feeder cells are non-proliferative.

19. A method according to claim 18 wherein said feeder cells are human fibroblasts.

20. A method to culture mammalian cells on a therapeutic vehicle comprising the steps of:
i) providing a preparation comprising a therapeutic vehicle comprising a substrate and attached thereto feeder cells; and cell culture media wherein said media does not include agents which promote or enhance the establishment of mammalian cells in culture;
ii) providing culture conditions which promote the production by said feeder cells of agents which promote mammalian cell culture; and
iii) adding to said preparation at least one mammalian cell the culturing of which is desired on said vehicle.

21. A method according to claim 20 wherein said mammalian cells are human.

22. A method according to claim 21 wherein said mammalian cells are selected form the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts,_oral mucosa keratinocytes, urethral fibroblasts and epithelial cells, bladder fibroblasts and epithelial cells, neuronal glial cells and neural cells, hepatocyte stellate cells and epithelial cells.

23. A method according to claim 22 wherein said mammalian cells are autologous.

24. A method according to claim 22 wherein said feeder cells are fibroblasts, preferably human fibroblasts.

25. A method according to claim 20 wherein said therapeutic vehicle is selected from the group consisting of: prosthesis; implant; matrix; stent; biodegradable matrix; polymeric film; bandages, gauze, plaster casts, tissue engineering scaffolds e.g. PGA/PLA scaffold.

26. A therapeutic vehicle obtainable by the method according to claim 20.

27. A cell culture vessel containing a mammalian cell culture obtainable by the method according to claim 1.

28. A method for the production of recombinant protein comprising:
i) providing a cell culture vessel comprising a cell culture support surface comprising feeder cells and cell culture media which does not include agents which promote or enhance the establishment of mammalian cells in culture;
ii) providing culture conditions which promote the production by said feeder cells or agents which promote mammalian cell culture; and
iii) adding to said vessel at least one transfected mammalian cell the culturing of which is produces said recombinant protein.

29. A method according to claim 27 wherein said recombinant protein is a therapeutic protein.

30. A method according to claim 28 wherein said therapeutic protein is a cytokine.

31. A method according to claim 30 wherein said cytokine is selected from the group consisting of: growth hormone; leptin; erythroprotein; prolactin; TNF, interleukins (IL), IL-2, IL-3, IL-4, IL-5, IL-6, IL-7, IL-9, IL-10, IL-11; and p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukemia inhibitory factor (LIF); oncostatin M (OSM); interferon, IFNα and IFNγ.

32. A method according to claim 28 wherein said therapeutic protein is an antigenic polypeptide.

33. A method according to claim 28 wherein said method further comprises the purification of said recombinant protein.

34. A recombinant protein obtained by the method according to claim 33.

35. A composition comprising a protein according to claim 34.
